(19) United States
(12) Patent Application Publication
(10) Pub. No.: US 2015/0087953 A1
(43) Pub. Date: Mar. 26, 2015

(54) NON-INVASIVE INTRAOCULAR PRESSURE SENSOR

(71) Applicant: National Chiao Tung University, Hsinchu City (TW)

(72) Inventors: Jin-Chern Chiou, Hsinchu City (TW); Tzu-Sen Yang, Hsinchu City (TW); Yu-Chieh Huang, Hsinchu City (TW); Kuan-Ting Yeh, Hsinchu City (TW)

(73) Assignee: National Chiao Tung University, Hsinchu City (TW)

(21) Appl. No.: 14/178,287

(22) Filed: Feb. 12, 2014

(30) Foreign Application Priority Data
Sep. 26, 2013 (TW) 102134839

(51) Int. Cl. A61B 3/16

(52) U.S. Cl. CPC A61B 3/16
USPC 600/398

(57) ABSTRACT

A non-invasive intraocular pressure sensor adapted to be configured on an eyeball is provided. The non-invasive intraocular pressure sensor includes a sensing unit and a readout circuit. The sensing unit includes a plurality of electrode layers and a dielectric layer. The dielectric layer encloses the electrode layers and fills therebetween, and the electrode layers and the dielectric layer form a capacitor. A variation of capacitance of the capacitor varies with a variation of an intraocular pressure of the eyeball. The readout circuit is electrically connected to the sensing unit.

### CROSS-REFERENCE TO RELATED APPLICATION

This application claims the priority benefit of Taiwan application serial no. 102134839, filed on Sep. 26, 2013. The entirety of the above-mentioned patent application is hereby incorporated by reference herein and made a part of this specification.

### BACKGROUND OF THE INVENTION

1. Field of the Invention

The invention relates to an intraocular pressure sensor and more particularly relates to a non-invasive intraocular pressure sensor.

2. Description of Related Art

With the development of the society, people are spending more and more time on working. The progress of technology also increases use of electronic products significantly. Long hours of work or use of electronic products at a close distance may result in overuse of the eyes and easily cause discomfort, such as eye fatigue and excessive intraocular, etc., which accelerates aging of the eye and easy causes high degree myopia. In general, people having high degree myopia, diabetes, or high blood pressure, or having a family history of glaucoma are at high risk of glaucoma, or even blindness. Therefore, it is a very important part to timely monitor intraocular pressure in order to maintain the health of the eyes.

Currently, the typical method for measurement of the intraocular pressure is to use an optical instrument or a piezoresistive tonometer to measure the patient's eye pressure when the patient comes to see the doctor. However, these two methods are limited by the clinic hours and are not suitable for long-term monitoring. In addition, there is another method for measuring the intraocular pressure, which is to implant a chip in the eyes of the patient so as to monitor for a long time. However, this method requires surgery. There are certain risks due to surgery, and thus the patient's acceptance is generally not high. In recent years, a resistive non-invasive intraocular pressure sensor has been developed, which includes a resistive element embedded in the contact lens and utilizes resistance variation caused by the variation of the intraocular pressure of the eyeball to measure the intraocular pressure. The advantage is that the intraocular pressure of the patient can be monitored for a long period of time without surgery. However, the resistance value has very little variation, and the frequency of variation in the intraocular pressure is 0.01 Hz or less. It can be known from the equation of noise power spectral density, namely V²=4 kTR (unit: V²/Hz, wherein k represents the Boltzmann constant, T represents the absolute temperature, and R represents the resistance value), that the noise is very large when the resistance and the frequency are both very small. Therefore, it is difficult to use this method to measure the correct values of intraocular pressure variation, and due to a large number of noise, the subsequent signal processing of the values also becomes difficult.

### SUMMARY OF THE INVENTION

The invention provides a non-invasive intraocular pressure sensor that is adapted for long-term monitoring and obtaining relatively stable intraocular pressure signals without surgery.

A non-invasive intraocular pressure sensor of the invention is adapted to be configured on an eyeball. The non-invasive intraocular pressure sensor includes a sensing unit and a readout circuit. The sensing unit includes a plurality of electrode layers and a dielectric layer. The dielectric layer encloses the electrode layers and fills between the electrode layers. The electrode layers and the dielectric layer form a capacitor, and a variation of capacitance of the capacitor varies with the variation of the intraocular pressure of the eyeball. The readout circuit is electrically connected to the sensing unit.

In an embodiment of the invention, the electrode layers include a first electrode layer and a second electrode layer electrically insulated from the first electrode layer.

In an embodiment of the invention, a material of the dielectric layer is a polymer material.

In an embodiment of the invention, the electrode layers include circular main body portions that share a center axis.

In an embodiment of the invention, the main body portions partially overlap each other in a front view thereof.

In an embodiment of the invention, the main body portions do not overlap each other in the front view thereof.

In an embodiment of the invention, each of the electrode layers further includes a plurality of protruding portions protruding from the main body portion.

In an embodiment of the invention, the protruding portions all protrude outward or inward, wherein the main body portions partially overlap each other and the protruding portions partially overlap each other.

In an embodiment of the invention, the electrode layers include a first electrode layer and a second electrode layer electrically insulated from the first electrode layer, wherein the first electrode layer includes a first main body portion and a plurality of first protruding portions protruding from the first main body portion, and the second electrode layer includes a second main body portion and a plurality of second protruding portions protruding from the second main body portion. The first protruding portions protrude toward the second main body portion while the second protruding portions protrude toward the first main body portion, and the first protruding portions and the second protruding portions are arranged alternately.

In an embodiment of the invention, the readout circuit converts the variation of the capacitance to a voltage signal.

In an embodiment of the invention, the readout circuit converts the variation of the capacitance to a digital signal.

In an embodiment of the invention, the readout circuit converts the variation of the capacitance to an oscillation frequency signal.

In an embodiment of the invention, the readout circuit includes an inductor, wherein the sensing unit and the inductor form an oscillation circuit.

In an embodiment of the invention, the readout circuit includes an inductor and a resistor, wherein the sensing unit, the inductor, and the resistor form an oscillation circuit.

In an embodiment of the invention, the non-invasive intraocular pressure sensor further includes a soft contact lens.

In an embodiment of the invention, the sensing unit and the readout circuit are embedded in the soft contact lens, and the sensing unit and the soft contact lens share a center axis.

In an embodiment of the invention, the readout circuit is embedded in the soft contact lens while the sensing unit is disposed on an external surface of the soft contact lens and shares a center axis with the soft contact lens.

In an embodiment of the invention, the non-invasive intraocular pressure sensor further includes a power supply unit electrically connected to the readout circuit.

In an embodiment of the invention, the non-invasive intraocular pressure sensor further includes a data conversion unit electrically connected to the readout circuit and the power supply unit.

In an embodiment of the invention, the non-invasive intraocular pressure sensor further includes a wireless transmission unit electrically connected to the readout circuit and the power supply unit.

In an embodiment of the invention, the non-invasive intraocular pressure sensor further includes a data conversion unit and a wireless transmission unit, wherein the data conversion unit is electrically connected to the readout circuit and the wireless transmission unit.

In an embodiment of the invention, a material of the first electrode layer and the second electrode layer is a metal, an alloy, or a combination of the above.

In an embodiment of the invention, a material of the first electrode layer and the second electrode layer is a metal oxide.

Based on the above, the non-invasive intraocular pressure sensor of the invention measures the intraocular pressure by detecting the variation of capacitance resulting from the variation of the intraocular pressure of the eyeball. According to the equation of noise power spectral density, it is known that the noise is inversely proportional to the capacitance. In other words, the higher the capacitance, the lower the noise. Therefore, the non-invasive intraocular pressure sensor of the invention is able to measure values of variation of the intraocular pressure with relatively low noise and high accuracy, and the values of variation of the intraocular pressure are relatively stable and suitable for the subsequent signal processing, which is conducive to improving the analytical ability of an intraocular pressure measuring system. In addition, because the non-invasive intraocular pressure sensor of the invention is a non-implanted intraocular pressure sensor, surgery is not required. Further, the combination of the non-invasive intraocular pressure sensor and the contact lens allows the user to wear the non-invasive intraocular pressure sensor by himself/herself and use it for a long period of time, which is suitable for long-term monitoring.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate exemplary embodiments of the invention and, together with the description, serve to explain the principles of the invention.

FIG. 1A is a schematic top view of a non-invasive intraocular pressure sensor according to the first embodiment of the invention.

FIG. 1B is a schematic cross-sectional view taken along the line section A-A′ in FIG. 1A.

FIG. 1C is a schematic cross-sectional view of another non-invasive intraocular pressure sensor according to the first embodiment of the invention.

FIG. 2 is a diagram illustrating the relationship between a curvature of a cornea and the variation of an intraocular pressure.

FIG. 3A and FIG. 3B are schematic top view and cross-sectional view of a non-invasive intraocular pressure sensor according to the second embodiment of the invention.

FIG. 4A and FIG. 4B are schematic top view and cross-sectional view of a non-invasive intraocular pressure sensor according to the third embodiment of the invention.

FIG. 5A and FIG. 5B are schematic top view and cross-sectional view of a non-invasive intraocular pressure sensor according to the fourth embodiment of the invention.

FIG. 6 is a diagram of a non-invasive intraocular pressure sensor according to the fifth embodiment of the invention.

FIG. 7 is a diagram of a non-invasive intraocular pressure sensor according to the sixth embodiment of the invention.

FIG. 8 is a diagram of a non-invasive intraocular pressure sensor according to the seventh embodiment of the invention.

FIG. 9 is a diagram of a non-invasive intraocular pressure sensor according to the eighth embodiment of the invention.

### DESCRIPTION OF THE EMBODIMENTS

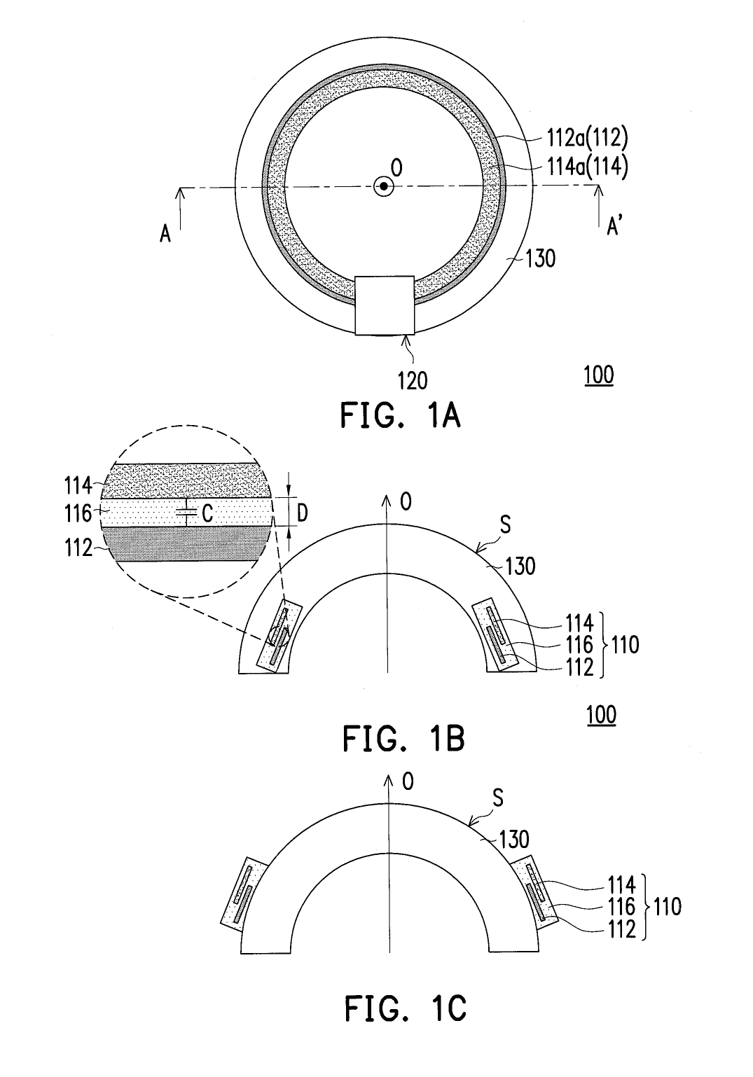

FIG. 1A is a schematic top view of a non-invasive intraocular pressure sensor according to the first embodiment of the invention. FIG. 1B is a schematic cross-sectional view taken along the line section A-A′ in FIG. 1A. FIG. 1C is a schematic cross-sectional view of another non-invasive intraocular pressure sensor according to the first embodiment of the invention. In FIG. 1A, a dielectric layer of a sensing unit is omitted. With reference to FIG. 1A and FIG. 1B, a non-invasive intraocular pressure sensor 100 of this embodiment is adapted to be configured on an eyeball of a user for measuring an intraocular pressure of the eyeball. The non-invasive intraocular pressure sensor 100 includes a sensing unit 110 and a readout circuit 120. The sensing unit 110 and the readout circuit 120 may be used with a flexible member. For example, the sensing unit 110 and the readout circuit 120 are disposed on or embedded in the flexible member to be worn or removed by the user easily.

More specifically, the non-invasive intraocular pressure sensor 100 may further include a soft contact lens 130. In this embodiment, the sensing unit 110 is embedded in the soft contact lens 130, for example, but the invention is not limited thereto. Moreover, the sensing unit 110 and the soft contact lens 130 share a center axis O, for example. In addition, the readout circuit 120 may be embedded in the soft contact lens 130, disposed on the soft contact lens 130, or connected with the soft contact lens 130 externally (that is, the readout circuit 120 is not in contact with the soft contact lens 130). For instance, the readout circuit 120 may be disposed on the face of the user or in other suitable positions and electrically connected with the sensing unit 110 via two wires.

To make it conformable to wear, the soft contact lens 130 is preferably formed using a hydrophilic material with high oxygen permeability such that the non-invasive intraocular pressure sensor 100 is adapted to be worn for a long period of time for long-term monitoring. For example, the material of the soft contact lens 130 can be hydrogel (scientific name: 2-Hydroxyethyl methacrylate, HEMA).

The sensing unit 110 includes a plurality of electrode layers and a dielectric layer 116. In the following descriptions of this embodiment, a first electrode layer 112 and a second electrode layer 114 are given as an example; however, the invention should not be construed as limited thereto. In some other embodiments, the sensing unit 110 may include two electrode layers or more. In this embodiment, the first electrode layer 112 has a first main body portion 112a that is circular, and the second electrode layer 114 has a second main body portion 114a that is circular, wherein the first main body portion 112a and the second main body portion 114a share the center axis O. In addition, the first main body portion 112a and the second main body portion 114a overlap each other partially, for example; however, the invention should not be construed as limited thereto.

The dielectric layer 116 encloses the first electrode layer 112 and the second electrode layer 114 and fills between the first electrode layer 112 and the second electrode layer 114, so as to electrically insulate the first electrode layer 112 and the second electrode layer 114 from each other. The dielectric layer 116 is formed using a polymer material, such as Parylene C, for example. The first electrode layer 112 and the second electrode layer 114 are formed using a metal, an alloy, or a combination thereof, for example; however, the invention should not be construed as limited thereto. In another embodiment, the first electrode layer 112 and the second electrode layer 114 are formed using a transparent conductive material, such as a metal oxide, to provide better light transmittance. The metal oxide may be indium tin oxide, indium zinc oxide, aluminum tin oxide, aluminum zinc oxide, indium germanium zinc oxide, other suitable oxides, or a stack layer including at least two of the above.

The first electrode layer 112, the second electrode layer 114, and the dielectric layer 116 disposed between the first electrode layer 112 and the second electrode layer 114 form a capacitor C, wherein a variation of capacitance of the capacitor C varies with the variation of the intraocular pressure of the eyeball. The readout circuit 120 is electrically connected to the sensing unit 110. Depending on different design requirements, the readout circuit 120 is adapted to convert the variation of capacitance to a voltage signal, digital signal or oscillation frequency signal to be analyzed and processed by a reader and a controller that are connected externally. In the case that the variation of capacitance is converted to an oscillation frequency signal, the readout circuit 120 may further include an inductor that is not illustrated here, wherein the sensing unit 110 and the inductor form an oscillation circuit. Alternatively, the readout circuit 120 may further include the inductor and a resistor that are not illustrated here, wherein the sensing unit 110, the inductor, and the resistor form an oscillation circuit.

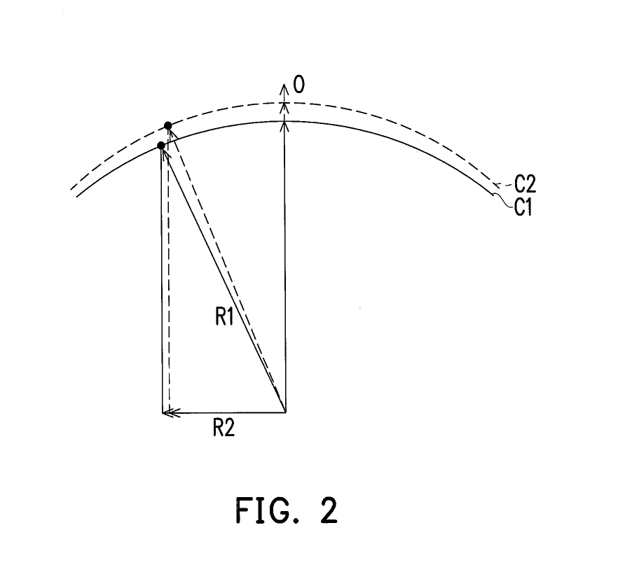

How the variation of the intraocular pressure causes the variation of capacitance is explained below with reference to FIG. 1B and FIG. 2. FIG. 2 is a diagram illustrating the relationship between a curvature of a cornea and the variation of the intraocular pressure. With reference to FIG. 1B and FIG. 2, when the non-invasive intraocular pressure sensor 100 is worn on the eyeball of the user, the non-invasive intraocular pressure sensor 100 is bent conform to the curvature of the cornea of the eyeball, as indicated by a curve C1 of FIG. 2. However, the curvature of the cornea varies with the variation of the intraocular pressure. For example, as the intraocular pressure increases, as indicated by a curve C2 of FIG. 2, a curvature radius R1 of the non-invasive intraocular pressure sensor 100 increases, and as a result, a projection radius R2 of the curvature radius R1 on a plane perpendicular to the center axis O decreases correspondingly. In other words, as the curvature of the cornea varies with the variation of the intraocular pressure, a degree of bending of the non-invasive intraocular pressure sensor 100 varies correspondingly. Accordingly, the first electrode layer 112 and the second electrode layer 114 may be deformed respectively. For example, the variation of the curvature of the cornea stretches the second electrode layer 114 that is disposed on an external side and compresses the first electrode layer 112 that is disposed on an internal side; or a distance D or an area between the first electrode layer 112 and the second electrode layer 114 varies with the variation of the curvature of the cornea, so that the variation of capacitance of the capacitor C varies with the variation of the intraocular pressure of the eyeball.

It is known from the equation of noise power spectral density, namely V²=kT/C (unit: V²/Hz, wherein k represents a Boltzmann constant, T represents an absolute temperature, and C represents the capacitance), that the higher the capacitance, the lower the noise. Therefore, in comparison with a resistive non-invasive intraocular pressure sensor, the non-invasive intraocular pressure sensor 100 of this embodiment is more suitable for measuring slight variation of the intraocular pressure. Because the non-invasive intraocular pressure sensor 100 of this embodiment is able to measure values of variation of the intraocular pressure with relatively low noise and high accuracy, the values of variation of the intraocular pressure are relatively stable and suitable for the subsequent signal processing, which is conducive to improving the analytical ability of an intraocular pressure measuring system.

It is worth mentioning that, as the curvature of the cornea varies with the variation of the intraocular pressure, the first electrode layer 112 and the second electrode layer 114 have the maximum deformation at an external surface of the soft contact lens 130. Thus, in another embodiment, as illustrated in FIG. 1C, a variation of the capacitance can be further increased by disposing the sensing unit 110 on an external surface S of the soft contact lens 130.

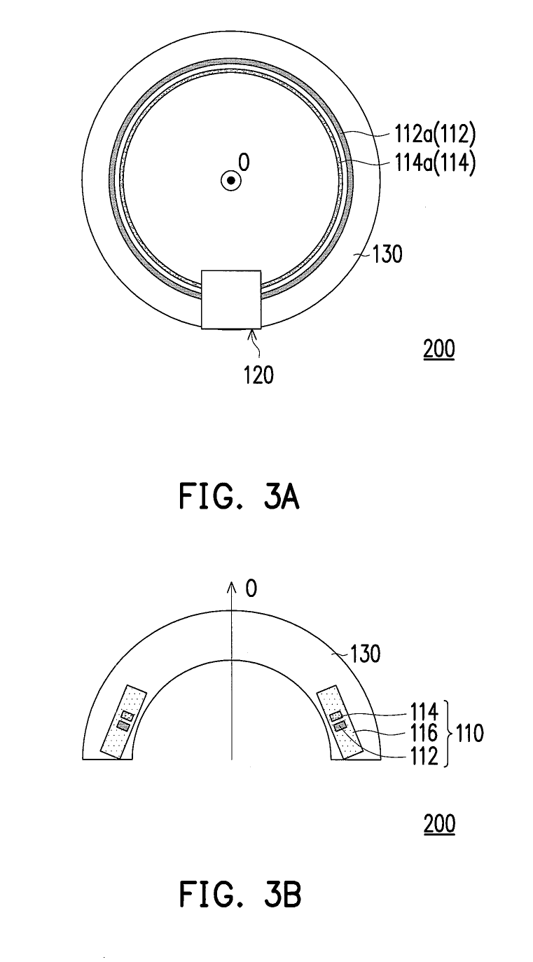

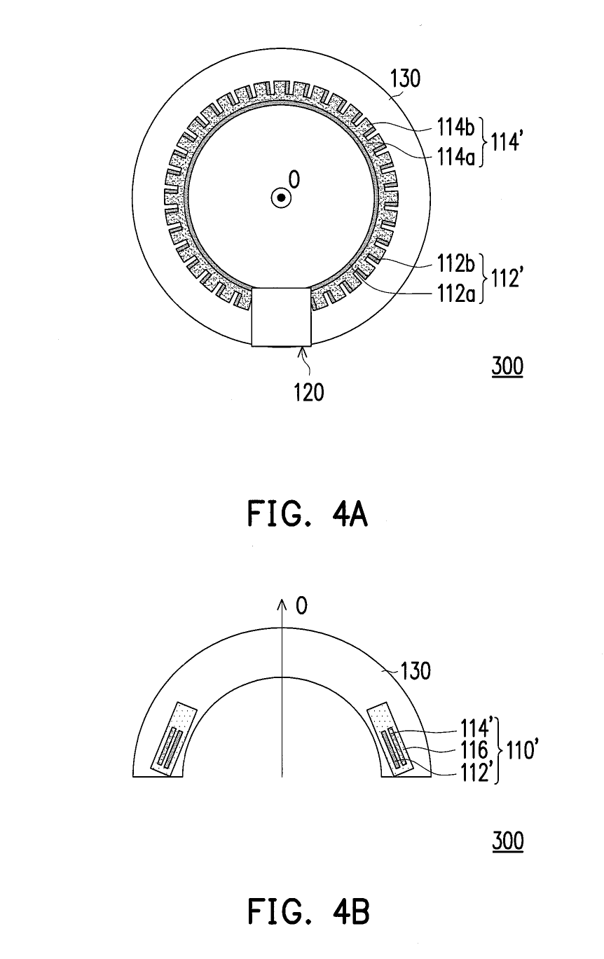

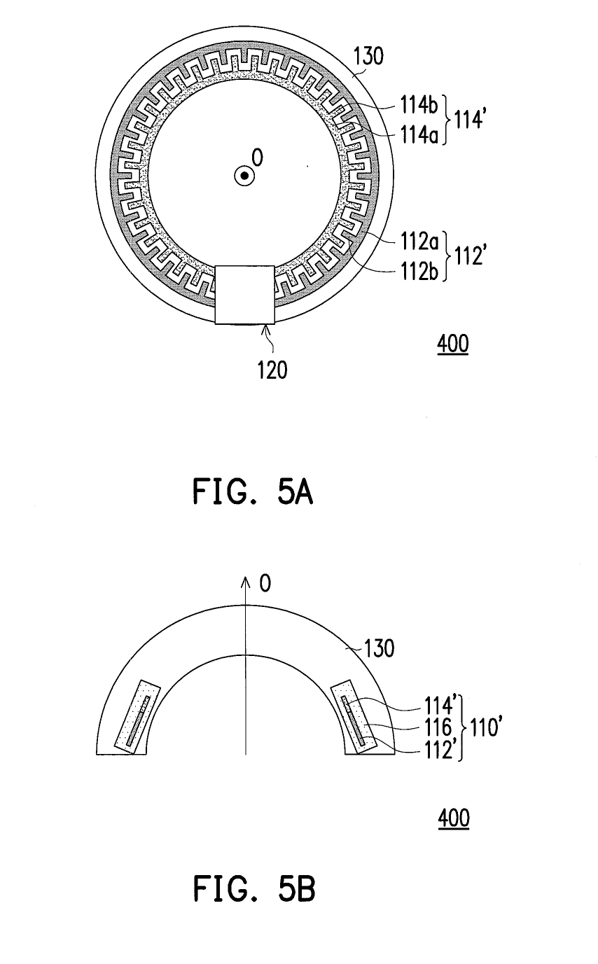

It should be noted that the pattern design and relative configuration of the first electrode layer 112 and the second electrode layer 114 of the invention are not limited to the examples illustrated in FIG. 1A to FIG. 1C. Please refer to FIG. 3A and FIG. 3B, FIG. 4A and FIG. 4B, and FIG. 5A and FIG. 5B for other embodiments of the first electrode layer 112 and the second electrode layer 114. FIG. 3A and FIG. 3B are schematic top view and cross-sectional view of a non-invasive intraocular pressure sensor according to the second embodiment of the invention. With reference to FIG. 3A and FIG. 3B, a non-invasive intraocular pressure sensor 200 of this embodiment is similar to the non-invasive intraocular pressure sensor 100 of FIG. 1A and FIG. 1B. Thus, identical elements are assigned with the same reference numerals. A main difference therebetween lies in that, in a front view as shown in FIG. 3A, the first main body portion 112a and the second main body portion 114a do not overlap each other.

FIG. 4A and FIG. 4B are schematic top view and cross-sectional view of a non-invasive intraocular pressure sensor according to the third embodiment of the invention. With reference to FIG. 4A and FIG. 4B, a non-invasive intraocular pressure sensor 300 of this embodiment is similar to the non-invasive intraocular pressure sensor 100 of FIG. 1A and FIG. 1B. Thus, identical elements are assigned with the same reference numerals. A main difference lies in that a first electrode layer 112′ of a sensing unit 110′ further includes a plurality of first protruding portions 112b connected with the first main body portion 112a, and a second electrode layer 114′ further includes a plurality of second protruding portions 114b connected with the second main body portion 114a. Moreover, the first main body portion 112a and the second main body portion 114a overlap each other partially while the first protruding portions 112b and the second protruding portions 114b overlap each other partially, and the first protruding portions 112b and the second protruding portions 114b all protrude outward, for example; however, the invention should not be construed as limited thereto. In another embodiment, the first protruding portions 112b and the second protruding portions 114b may all protrude inward.

FIG. 5A and FIG. 5B are schematic top view and cross-sectional view of a non-invasive intraocular pressure sensor according to the fourth embodiment of the invention. With reference to FIG. 5A and FIG. 5B, a non-invasive intraocular pressure sensor 400 of this embodiment is similar to the non-invasive intraocular pressure sensor 300 of FIG. 4A and FIG. 4B. Thus, identical elements are assigned with the same reference numerals. A main difference therebetween lies in that the first protruding portions 112b protrude toward the second main body portion 114a while the second protruding portions 114b protrude toward the first main body portion 112a, and the first protruding portions 112b and the second protruding portions 114b are arranged alternately without overlapping each other. It should be understood that, under the aforementioned concept, the shapes and sizes of the patterns of the first main body portion 112a, the second main body portion 114a, the first protruding portion 112b, and the second protruding portion 114b may be varied in accordance with the design requirements, and details thereof will omitted here.

Hereinafter, FIG. 6 to FIG. 9 illustrate an intraocular pressure measuring system adapted to use the non-invasive intraocular pressure sensor 100, 200, 300, or 400. FIG. 6 is a diagram of a non-invasive intraocular pressure sensor according to the fifth embodiment of the invention. With reference to FIG. 6, a non-invasive intraocular pressure sensor 500 of this embodiment includes a sensing unit 510 and the readout circuit 120, wherein the sensing unit 510 may be the sensing unit 110 or 110′ illustrated in FIG. 1A, FIG. 1B, FIG. 1C, FIG. 3A, FIG. 3B, FIG. 4A, FIG. 4B, FIG. 5A, or FIG. 5B.

In addition, the non-invasive intraocular pressure sensor 500 may further include a power supply unit 140, such as a low dropout regulator, which is electrically connected to the readout circuit 120. Furthermore, by electrically connecting the power supply unit 140 and the readout circuit 120 to a reader 610 and electrically connecting the reader 610 to a controller 620, the aforementioned voltage signal, digital signal, or oscillation frequency signal can be analyzed and processed. For instance, in the case that the readout circuit 120 converts the variation of capacitance to a voltage signal, the reader 610 may include an analog to digital converter (ADC); in the case that the readout circuit 120 converts the variation of capacitance to a digital signal, the reader 610 may include a digital filter; and in the case that the readout circuit 120 converts the variation of capacitance to an oscillation frequency signal, the reader 610 may include a digital frequency converter. The controller 620 is a digital signal processor or a micro processor, for example. Moreover, the controller 620 may be coupled to a storage unit or an instant monitoring system (e.g. medical station) that is not illustrated here.

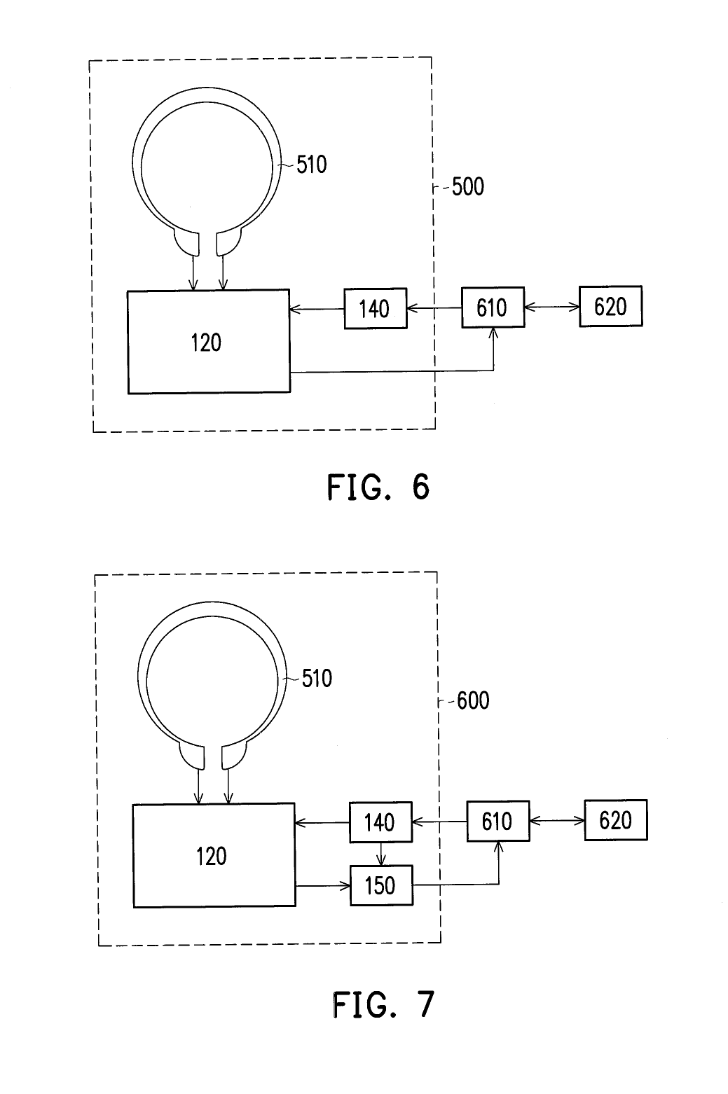

FIG. 7 is a diagram of a non-invasive intraocular pressure sensor according to the sixth embodiment of the invention. With reference to FIG. 7, a non-invasive intraocular pressure sensor 600 of this embodiment is similar to the non-invasive intraocular pressure sensor 500 of FIG. 6. Thus, identical elements are assigned with the same reference numerals and will not be described in detail hereinafter. A main difference lies in that the non-invasive intraocular pressure sensor 600 of this embodiment further integrates a data conversion unit 150 in the non-invasive intraocular pressure sensor 600. More specifically, the non-invasive intraocular pressure sensor 600 includes the data conversion unit 150 that is electrically connected to the readout circuit 120 and the power supply unit 140, and the data conversion unit 150 is electrically connected with the reader 610.

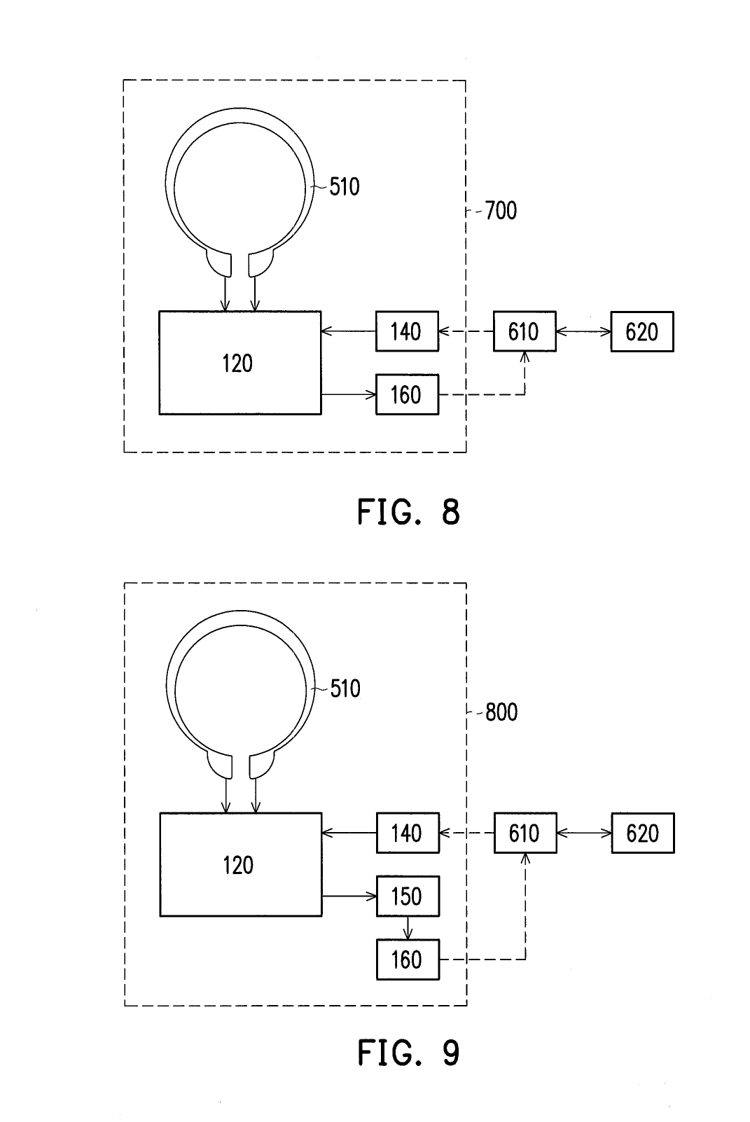

FIG. 8 is a diagram of a non-invasive intraocular pressure sensor according to the seventh embodiment of the invention. With reference to FIG. 8, a non-invasive intraocular pressure sensor 700 of this embodiment is similar to the non-invasive intraocular pressure sensor 500 of FIG. 6. Thus, identical elements are assigned with the same reference numerals and will not be described in detail hereinafter. A main difference lies in that the non-invasive intraocular pressure sensor 700 of this embodiment transmits a signal to the reader 610 via wireless transmission, and the reader 610 is power-supplied by the power supply unit 140 via wireless transmission. More specifically, the non-invasive intraocular pressure sensor 700 includes a wireless transmission unit 160 that is electrically connected to the readout circuit 120 and the power supply unit 140, and the wireless transmission unit 160 is coupled to the reader 610. The wireless transmission unit 160 may be a radio frequency identification (RFID) system. Moreover, the wireless transmission unit 160 of this embodiment may include a circular antenna, which is embedded in or disposed on the soft contact lens, for example. In addition, the circular antenna surrounds outside the sensing unit 510 and share a center axis with the sensing unit 510, for example.

FIG. 9 is a diagram of a non-invasive intraocular pressure sensor according to the eighth embodiment of the invention. With reference to FIG. 9, a non-invasive intraocular pressure sensor 800 of this embodiment is similar to the non-invasive intraocular pressure sensor 700 of FIG. 8. Thus, identical elements are assigned with the same reference numerals and will not be described in detail hereinafter. A main difference lies in that the non-invasive intraocular pressure sensor 800 of this embodiment further includes the data conversion unit 150, wherein the data conversion unit 150 is electrically connected to the readout circuit 120 and the wireless transmission unit 160. That is, the wireless transmission unit 160 transmits signals processed by the data conversion unit 150 to the reader 610 via wireless transmission.

To conclude the above, the non-invasive intraocular pressure sensor of the invention measures the intraocular pressure by detecting the variation of capacitance resulting from the variation of the intraocular pressure of the eyeball. According to the equation of noise power spectral density, it is known that the noise is inversely proportional to the capacitance. In other words, the higher the capacitance, the lower the noise. Therefore, the non-invasive intraocular pressure sensor of the invention is able to measure values of variation of the intraocular pressure with relatively low noise and high accuracy, and the values of variation of the intraocular pressure are relatively stable and suitable for the subsequent signal processing, which is conducive to improving the analytical ability of the intraocular pressure measuring system. In addition, because the non-invasive intraocular pressure sensor of the invention is a non-implanted intraocular pressure sensor, surgery is not required. The combination of the non-invasive intraocular pressure sensor and the contact lens allows the user to wear the non-invasive intraocular pressure sensor by himself/herself and use it for a long period of time, which is suitable for long-term monitoring.

It will be apparent to those skilled in the art that various modifications and variations can be made to the disclosed embodiments without departing from the scope or spirit of the invention. In view of the foregoing, it is intended that the invention covers modifications and variations of this disclosure provided that they fall within the scope of the following claims and their equivalents.

What is claimed is:

1. A non-invasive intraocular pressure sensor adapted to be disposed on an eyeball, the non-invasive intraocular pressure sensor comprising:
a sensing unit comprising a plurality of electrode layers and a dielectric layer that encloses the electrode layers and fills between the electrode layers, wherein the electrode layers and the dielectric layer form a capacitor, and a variation of capacitance of the capacitor varies with a variation of an intraocular pressure of the eyeball; and
a readout circuit electrically connected to the sensing unit.

2. The non-invasive intraocular pressure sensor according to claim 1, wherein the electrode layers comprise a first electrode layer and a second electrode layer that is electrically insulated from the first electrode layer.

3. The non-invasive intraocular pressure sensor according to claim 1, wherein a material of the dielectric layer comprises a polymer material.

4. The non-invasive intraocular pressure sensor according to claim 1, wherein each of the electrode layers comprises a circular main body portion that shares a center axis with each other.

5. The non-invasive intraocular pressure sensor according to claim 4, wherein the main body portions partially overlap each other in a front view thereof.

6. The non-invasive intraocular pressure sensor according to claim 4, wherein the main body portions do not overlap each other in the front view thereof.

7. The non-invasive intraocular pressure sensor according to claim 4, wherein each of the electrode layers further comprises a plurality of protruding portions protruding from the main body portion.

8. The non-invasive intraocular pressure sensor according to claim 7, wherein the protruding portions all protrude outward or inward, and the main body portions partially overlap each other and the protruding portions partially overlap each other.

9. The non-invasive intraocular pressure sensor according to claim 7, wherein the electrode layers comprise a first electrode layer and a second electrode layer that is electrically insulated from the first electrode layer, wherein the first electrode layer comprises a first main body portion and a plurality of first protruding portions protruding from the first main body portion, and the second electrode layer comprises a second main body portion and a plurality of second protruding portions protruding from the second main body portion, wherein the first protruding portions protrude toward the second main body portion while the second protruding portions protrude toward the first main body portion, and the first protruding portions and the second protruding portions are arranged alternately.

10. The non-invasive intraocular pressure sensor according to claim 1, wherein the readout circuit converts the variation of the capacitance to a voltage signal.

11. The non-invasive intraocular pressure sensor according to claim 1, wherein the readout circuit converts the variation of the capacitance to a digital signal.

12. The non-invasive intraocular pressure sensor according to claim 1, wherein the readout circuit converts the variation of the capacitance to an oscillation frequency signal.

13. The non-invasive intraocular pressure sensor according to claim 12, wherein the readout circuit comprises an inductor, and the sensing unit and the inductor form an oscillation circuit.

14. The non-invasive intraocular pressure sensor according to claim 12, wherein the readout circuit comprises an inductor and a resistor, wherein the sensing unit, the inductor, and the resistor form an oscillation circuit.

15. The non-invasive intraocular pressure sensor according to claim 1, further comprising a soft contact lens.

16. The non-invasive intraocular pressure sensor according to claim 15, wherein the sensing unit is embedded in the soft contact lens and shares a center axis with the soft contact lens.

17. The non-invasive intraocular pressure sensor according to claim 15, wherein the sensing unit is disposed on an external surface of the soft contact lens and shares a center axis with the soft contact lens.

18. The non-invasive intraocular pressure sensor according to claim 1, further comprising a power supply unit electrically connected to the readout circuit.

19. The non-invasive intraocular pressure sensor according to claim 18, further comprising a data conversion unit electrically connected to the readout circuit and the power supply unit.

20. The non-invasive intraocular pressure sensor according to claim 18, further comprising a wireless transmission unit electrically connected to the readout circuit and the power supply unit.

21. The non-invasive intraocular pressure sensor according to claim 18, further comprising a data conversion unit and a wireless transmission unit, wherein the data conversion unit is electrically connected to the readout circuit and the wireless transmission unit.

22. The non-invasive intraocular pressure sensor according to claim 1, wherein a material of the first electrode layer and the second electrode layer comprises a metal, an alloy, or a combination of the above.

23. The non-invasive intraocular pressure sensor according to claim 1, wherein the material of the first electrode layer and the second electrode layer comprises a metal oxide.
